(19) United States
(12) Patent Application Publication
(10) Pub. No.: US 2021/0407646 A1
(43) Pub. Date: Dec. 30, 2021

(54) METHOD, DEVICE AND SYSTEM FOR PREDICTING AN EFFECT OF ACOUSTIC STIMULATION OF THE BRAIN WAVES OF AN INDIVIDUAL

(71) Applicant: DREEM, Paris (FR)

(72) Inventors: Valentin THOREY, Paris (FR); Clémence PINAUD, Paris (FR)

(21) Appl. No.: 17/290,025

(22) PCT Filed: Oct. 16, 2019
(86) PCT No.: PCT/FR2019/052440

§ 371 Date: Apr. 29, 2021

(30) Foreign Application Priority Data
Oct. 30, 2018 (FR) 18 60050

(51) Int. Cl. G16H 20/30; A61H 23/02; G16H 50/20

(52) U.S. Cl. CPC G16H 20/30; A61H 23/02; A61H 2201/501; A61H 2205/02; G16H 50/20

(57) ABSTRACT

A method, implemented by computer, for predicting an effect of an acoustic stimulation of the brain waves of an individual, the method including: acquisition of at least one measurement signal representative of a physiological signal of the individual, by a device for acoustic stimulation of brain waves that is suitable for being worn by the individual; analysis of the measurement signal by an artificial intelligence trained to predict the effect of an acoustic stimulation; and determination of whether an acoustic stimulation is to be performed by the device.

The invention relates to methods and devices for predicting an effect of an acoustic stimulation of the brain waves of an individual during sleep.

Currently, there are methods for stimulating the brain waves of an individual, in particular during different sleep phases of the individual.

Generally, such methods use an acoustic stimulation of brain waves to promote the generation of slow brain waves during deep sleep.

In particular, the individual's encephalogram can be analyzed to determine whether the individual has reached a stage of deep sleep, and is therefore in a state of sleep conducive to stimulation.

However, such methods are not sufficiently precise. Indeed, only the individual's encephalogram is taken into account. Acoustic stimulation thus may not cause any generation of slow waves, or may even wake the individual.

There is therefore a real need to develop a method for ensuring that an acoustic stimulation will be beneficial to the individual's sleep, for example by generating slow waves, before performing this stimulation.

The present invention improves the situation.

To this end, it proposes a method, implemented by computer means, for predicting an effect of an acoustic stimulation of the brain waves of an individual, the method comprising the following steps:
acquisition of at least one measurement signal, representative of a physiological signal of the individual, by a device for acoustic stimulation of brain waves that is suitable for being worn by the individual,
analysis of said measurement signal by an artificial intelligence trained to predict the effect of an acoustic stimulation, and
determination of whether an acoustic stimulation is to be performed by the device.

By means of these arrangements, the implemented method makes it possible to perform acoustic stimulations only when the impact of this stimulation will be positive for the individual. The use of artificial intelligence also makes it possible to obtain an exact prediction.

The embodiments described below can be combined with one another.

According to one embodiment, the artificial intelligence is a neural network, the method including a prior learning step comprising:
a plurality of successive acoustic stimulations of the brain waves of said individual,
an acquisition of measurement signals representative of a physiological signal of said individual at least before and after each of the acoustic stimulations of said plurality of acoustic stimulations,
for each of the acoustic stimulations, determination of a change in the measurement signals acquired after an acoustic stimulation in comparison to the measurement signal acquired before this acoustic stimulation, and association of an effect with said change,
training of said neural network until a threshold of convergence is reached, and
storage of said neural network.

Learning by the neural network is based on measurement signals acquired on the individual himself. The learning is therefore personalized, which ensures that the result of the prediction is correct.

According to one embodiment, the learning step further comprises the indication of physiological data of said individual, the neural network being further trained to predict the effect of an acoustic stimulation on the basis of said physiological data of said individual.

The physiological data make it possible to take into account general data of the individual and not only local data (namely the measurement signals). Unmeasurable physiological data can thus be taken into account to improve the robustness of the method.

According to one embodiment, the artificial intelligence is a neural network, the method including a prior learning step comprising:
at least one acoustic stimulation of the brain waves of a plurality of individuals,
an acquisition of measurement signals representative of a physiological signal of said plurality of individuals at least before and after said acoustic stimulation,
determination of a change in the measurement signals acquired after said acoustic stimulation in comparison to the measurement signals acquired before said acoustic stimulation, and association of an effect with said change,
training of said neural network until a threshold of convergence is reached, and
storage of said neural network.

The learning can thus also be done on the basis of measurement signals acquired from a plurality of individuals. This allows increasing the speed of convergence of the neural network since it is supplied with more data in less time than with a single individual. Also, since the measurement signals are acquired from a plurality of different individuals, the robustness of the neural network is increased.

According to one variant, during the learning step, the acoustic stimulations of the brain waves are imperceptible.

The neural network is thus more robust, since the learning takes place by analyzing signals following two types of stimulation: perceptible and imperceptible. This helps to identify when a change in the individual's signals is due to stimulation or to their natural sleep cycle.

According to one embodiment, the learning step further comprises the indication of physiological data of said plurality of individuals, the neural network being further trained to predict the effect of an acoustic stimulation on the basis of said physiological data of said plurality of individuals.

Similarly, non-measurable data can be taken into account. This further increases the robustness of the prediction method.

According to one embodiment, measurement signals representative of a physiological signal of said individual are acquired continuously, the neural network also being trained continuously and in real time to predict the effect of an acoustic stimulation on the basis of said measurement signals of said individual.

In this manner, the neural network can continuously be learning, which allows it to be personalized according to the individual using the device.

According to one embodiment, the artificial intelligence predicts an effect of an acoustic stimulation by analyzing sections of the measurement signals representative of a physiological signal of said individual, acquired over a sliding window of time.

The artificial intelligence is thus able to predict an effect of an acoustic stimulation by analyzing only a section of the measurement signal.

According to one embodiment, said section is acquired during an acquisition duration of between 10 seconds and one minute, and preferably about 30 seconds.

The measurement signal section to be analyzed is therefore sufficiently large to effectively predict the effect of an acoustic stimulation. In addition, the window is sufficiently small for the duration of analysis of the acquired signals to be fast.

According to one embodiment, an acoustic stimulation comprises the emission of an acoustic signal, the method further comprising a changing of at least one parameter of the acoustic signal to be emitted, on the basis of a result of the prediction by the artificial intelligence of the effect of an acoustic stimulation.

The method therefore makes it possible, in addition to predicting an effect of the acoustic stimulation, to adjust the parameters of the acoustic stimulation in order to generate a positive effect.

According to one embodiment, the effect comprises one or more of the following:
an awakening of the individual,
a non-awakening of the individual,
a changing of the sleep phase of the individual.

The effects induced by stimulation can therefore be predicted exhaustively by the method.

According to one embodiment, the physiological signal comprises at least one or more of the following:
a physiological electrical signal of the electroencephalogram, electrooculogram, or electrocardiogram type,
a cardiac activity signal,
a breath,
a movement.

The invention also relates to a device for predicting an effect of an acoustic stimulation of the brain waves of an individual, comprising:
acquisition means for acquiring at least one measurement signal representative of a physiological signal of the individual,
a processor communicating with the acquisition means and adapted to analyze said at least one measurement signal representative of a physiological signal of the individual, by an artificial intelligence trained to predict the effect of an acoustic stimulation.

In one embodiment, the device further comprises emission means designed to emit an acoustic signal audible to the individual, and communicating with said processor, said acoustic signal being emitted or not emitted depending on a result of the prediction by the artificial intelligence of the effect of said acoustic stimulation.

According to one embodiment, the artificial intelligence comprises a neural network trained to predict the effect of an acoustic stimulation and the device for acoustic stimulation further comprises a memory storing said neural network.

The invention also relates to a system for predicting an effect of an acoustic stimulation of the brain waves of an individual, comprising:
a device for predicting an effect of an acoustic stimulation of the brain waves of an individual, according to the invention,
a server that is remote from the device.

According to one embodiment, the server is capable of storing a database comprising a plurality of measurement signals representative of a physiological signal of at least one individual, said plurality of measurement signals having been acquired by said device.

According to one embodiment, the system comprises a plurality of devices, the plurality of devices being in communication with the server.

This makes it possible to acquire the measurement signals from a plurality of individuals, and to give the neural network access to them.

The invention also relates to a device for predicting an effect of an acoustic stimulation of the brain waves of an individual, the device being able to be worn by said individual and comprising:
acquisition means for acquiring at least one measurement signal representative of a physiological signal of the individual,
a processor comprising an artificial intelligence capable of analyzing said measurement signal and trained to predict the effect of an acoustic stimulation, the processor being able to determine whether an acoustic stimulation should be carried out.

The device further comprises emission means capable of carrying out a plurality of successive acoustic stimulations of the brain waves of said individual, the acquisition means being configured to acquire measurement signals representative of a physiological signal of said individual at least before and after each of the acoustic stimulations of said plurality of acoustic stimulations, the neural network being configured to be trained with at least said measurement signals acquired before and after each acoustic stimulation so as to determine a change in the measurements signals acquired after an acoustic stimulation in comparison to the measurement signal acquired before this acoustic stimulation and to associate an effect with said change, the device further comprising a memory capable of storing the neural network when said neural network has reached a predefined threshold of convergence.

The device comprises a user input module configured to enable the individual to enter physiological data, the neural network being configured to be supplied with said physiological data of said individual and to predict the effect of an acoustic stimulation on the basis of said physiological data of said individual.

According to one embodiment, the artificial intelligence is a neural network, the device further comprising emission means capable of performing at least one acoustic stimulation of the brain waves of a plurality of individuals, the acquisition means being configured to acquire, for each individual of the plurality of individuals, measurement signals representative of a physiological signal of said individual at least before and after each of the acoustic stimulations of said plurality of acoustic stimulations, the neural network being configured to be trained with at least said measurement signals acquired before and after each acoustic stimulation so as to determine a change in the measurement signals acquired after an acoustic stimulation in comparison to the measurement signal acquired before this acoustic stimulation and to associate an effect with said change, the device further comprising a memory capable of storing the neural network when said neural network has reached a predefined threshold of convergence.

According to one embodiment, the neural network is further configured to be supplied with physiological data of said plurality of individuals, the neural network being able to predict the effect of an acoustic stimulation on the basis of said physiological data of said plurality of individuals.

According to one embodiment, the acquisition means are configured to acquire, continuously, measurement signals representative of a physiological signal of said individual, the neural network also being trained continuously and in real time in order to predict the effect of an acoustic stimulation on the basis of said measurement signals from said individual.

According to one embodiment, the acquisition means are capable of acquiring a plurality of sections of the measurement signals representative of a physiological signal of said individual, each section being acquired over a sliding window of time and analyzed by the artificial intelligence in order to predict an effect of an acoustic stimulation.

According to one embodiment, said section is acquired during an acquisition duration of between 10 seconds and one minute, and preferably about 30 seconds.

According to one embodiment, an acoustic stimulation comprises the emission of an acoustic signal, the device comprising an electrical control unit adapted to modify at least one parameter of the acoustic signal to be emitted according to a result of the prediction of the effect of the acoustic stimulation by the artificial intelligence.

According to one embodiment, the effect comprises one or more of the following:
an awakening of the individual,
a non-awakening of the individual,
a changing of the sleep phase of the individual.

According to one embodiment, the physiological signal comprises at least one or more of the following:
a physiological electrical signal of the electroencephalogram, electrooculogram, or electrocardiogram type,
a cardiac activity signal,
a breath,
a movement.

Other features and advantages of the invention will be apparent from reading the following detailed description of some exemplary embodiments of the invention, and from examining the appended drawings in which:

FIG. 1 is a schematic view of a device for predicting an effect of an acoustic stimulation of the brain waves of an individual, according to one embodiment of the invention,

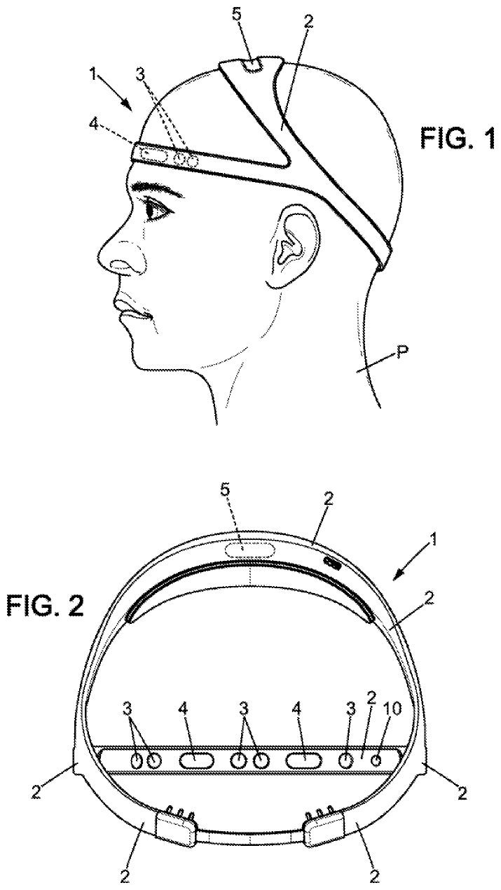

FIG. 2 is a schematic view of a device for predicting an effect of an acoustic stimulation of the brain waves of an individual, in another view,

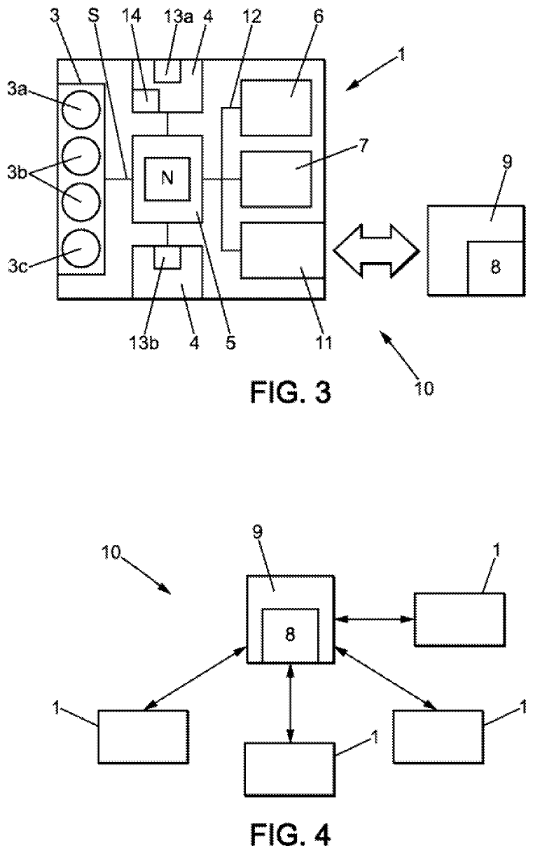

FIG. 3 is a block diagram of a system comprising a device according to one embodiment of the invention,

FIG. 4 is a block diagram of a system comprising a plurality of devices according to one embodiment of the invention,

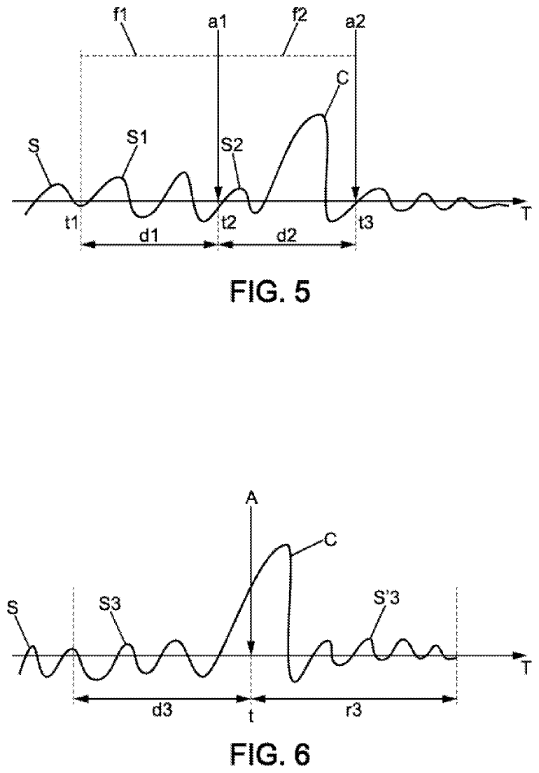

FIG. 5 illustrates the acquisition and analysis of sections of a measurement signal representative of a physiological signal of an individual,

FIG. 6 illustrates the teaching of the artificial intelligence on the basis of the acquisition and analysis of a measurement signal of the individual,

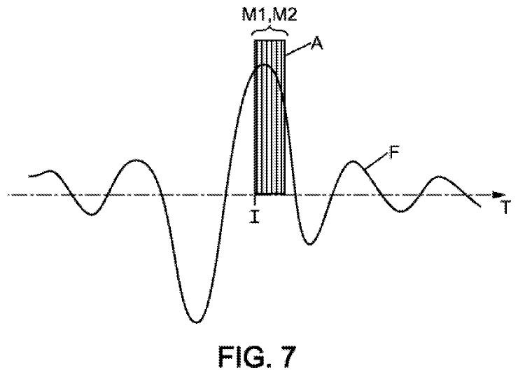

FIG. 7 illustrates a temporal form of a slow brain wave, an acoustic signal, and predefined temporal patterns according to one exemplary embodiment of the invention.

In the various figures, the same references designate identical or similar elements.

FIGS. 1 to 3 more specifically illustrate a device 1 for predicting an effect of an acoustic stimulation of the brain waves of an individual, or “device 1”.

“Effect” or “event” or “impact” are understood to mean a reaction or absence of reaction to the acoustic stimulation, by the individual P. More specifically, the effect or event or impact induces a change or absence of change in the brain waves of the individual P after the acoustic stimulation.

The device 1 is suitable for implementing a method for predicting an effect of an acoustic stimulation of the brain waves of an individual.

The device 1 is suitable for being worn by an individual P, for example during a sleep period of the individual P.

The device 1 is for example able to be worn on the head of the individual P.

To this end, the device 1 may include one or more support members 2 capable of at least partially surrounding the head of the individual P, so as to be held thereon. The support members 2 are for example in the form of one or more arms which can be arranged so as to surround the head of the individual P in order to hold the device 1 thereon.

The device 1 also comprises acquisition means 3 for acquiring at least one measurement signal, emission means 4 designed to emit an acoustic signal audible to the individual P, and at least one processor 5 comprising an artificial intelligence capable of analyzing the measurement signal. The artificial intelligence is, for example, a neural network trained to predict an effect of an acoustic stimulation of the brain waves of an individual.

The neural network may be stored in a memory 6 of the device 1.

The acquisition means 3, the emission means 4, the processor 5, and the memory 6 enable the device 1 to implement an acoustic stimulation of the brain waves of the individual P.

The acoustic stimulation of the brain waves of the individual P may be repeated one or more times.

The stimulation can thus be repeated a plurality of times by the device 1 during a period of operation of the device 1, for example during a sleep period of the individual P.

Such a period of operation of the device 1 may last for a duration of several hours, for example at least eight hours, in other words approximately one night of sleep.

In one embodiment of the invention, the device 1 is capable of implementing the stimulation autonomously. The device 1 may thus comprise a battery 7. The battery 7 may be mounted on the support member 2. The battery 7 is preferably capable of supplying energy for a duration of several hours without recharging, preferably at least eight hours so as to cover an average sleep period of an individual P.

“Autonomous” is understood to mean that the device 1 can operate for an extended period, preferably several hours, in particular at least eight hours, without needing to be recharged with electrical energy, to communicate with outside elements or to be structurally connected to an external device such as an attachment member such as an arm or a bracket.

In this manner, the device 1 is capable of being used in the daily life of an individual P without imposing any particular constraints.

To allow implementing the acoustic stimulation, the acquisition means 3, the emission means 4, the processor 5, and the memory 6 are also operably linked together and are capable of exchanging information and commands.

To this end, the acquisition means 3, the emission means 4, the processor 5, and the memory 6 may be mounted on the support member 2.

The memory 6 is capable of storing data which will be further detailed in the following description and may comprise at least one of the following elements: a measurement signal S acquired by the acquisition means 3, a neural network N able to analyze the measurement signal.

The measurement signal S may in particular be representative of a physiological electrical signal from the individual P.

The physiological electrical signal may for example include an electroencephalogram (EEG), an electromyogram (EMG), an electrooculogram (EOG), an electrocardiogram (ECG), or any other biosignal that is measurable on the individual P.

To this end, the acquisition means 3 comprise for example a plurality of electrodes 3 able to be in contact with the individual P, and in particular with the skin of the individual P, in order to acquire at least one measurement signal S representative of a physiological electrical signal from the individual P.

The physiological electrical signal advantageously includes an electroencephalogram (EEG) of the individual P.

To this end, the device 1 may comprise at least two electrodes 3 which include at least one reference electrode 3a and at least one EEG measurement electrode 3b. The device 1 may also include a ground electrode 3c.

The EEG measurement electrodes 3b are for example arranged on the surface of the scalp of the individual P.

In other embodiments, the device 1 may further comprise an EMG measurement electrode and an EOG measurement electrode.

The measurement electrodes 3 may be dry electrodes or electrodes covered with a contact gel. The electrodes 3 may also be textile or silicone electrodes.

The acquisition means 3 may also include devices for acquiring measurement signals S which are not only electrical.

A measurement signal S can thus be representative of a physiological signal of the individual P.

The measurement signal S may in particular be representative of a non-electrical or not fully electrical physiological signal of the individual P, for example a cardiac activity signal such as a heart rate, a breath of the individual P, or movements of the individual P.

To this end, the acquisition means 3 may include a heart rate detector, a breathing detector, an accelerometer, a bioimpedance sensor, or a microphone.

The acquisition means 3 may also include devices for acquiring measurement signals S representative of the individual's environment.

For example, the measurement signal S is representative of an air quality around the individual P, for example a carbon dioxide level, a temperature, or an ambient noise level.

Finally, the acquisition means 3 may include a user input module enabling the individual P to enter physiological data such as age, the quality of his night, the type of sleeper that the individual considers himself to be.

The measurement signal S can then be representative of a physiological data item of the individual P.

In one embodiment of the invention, the measurement signal S may undergo processing.

The processing of the measurement signal S may comprise, for example, at least one of the following:
frequency filtering,
amplification of the measurement signal S,
sampling of the measurement signal S by means of an analog-to-digital converter.

Thus, the acquisition means 3 may include an analog or digital module. Alternatively, the electrodes 3 are capable of carrying out one of the above preprocessing.

The processed or unprocessed measurement signals S can be sent to the processor 5 so that the artificial intelligence is able to predict the effect of an acoustic stimulation of the brain waves of the individual P.

In particular, during the sleep period of the individual P, the individual P may be subjected to several acoustic stimulations. More specifically, the acoustic stimulations are emitted when the individual is in a state of sleep called “deep sleep”.

The artificial intelligence may in particular be configured to analyze these measurement signals according to a sliding window of time whose size corresponds to a duration of acquisition of a section s of the measurement signal S to be analyzed.

In other words, the measurement signals are temporally sliced. Each section s of the measurement signal S represents a portion of the measurement signal acquired during an acquisition duration. This acquisition duration may in particular be between ten seconds and one minute, and preferably may be about thirty seconds.

According to an embodiment illustrated in FIG. 5, the sliding window, defining in particular a time of a potential stimulation, slides as a function of the acquisition duration.

More precisely, at time t1, a section s1 of the measurement signal S is acquired by the acquisition means 3 during acquisition duration d1 of sliding window f1. At the end of acquisition duration d1, section s1 of the measurement signal is analyzed by the artificial intelligence to determine whether an acoustic stimulation can be performed at moment a1 of the potential acoustic stimulation for signal section s1.

Then, at time t2, a section s2 of the measurement signal S is acquired by the acquisition means 3 during acquisition duration d2 of sliding window f2. At the end of acquisition duration d2, section s2 of the measurement signal is analyzed by the artificial intelligence to determine whether an acoustic stimulation can be performed at moment a2 of the potential acoustic stimulation for signal section s2.

The sliding window therefore slides from acquisition duration to acquisition duration. For example, the sliding window slides by thirty seconds every thirty seconds. The sections s of the measurement signal S to be analyzed thus do not overlap.

Alternatively, the sliding window slides according to a shorter time interval. For example, the sliding window slides by one second every second or by five seconds every five seconds. In this manner, the sections s of the measurement signal S to be analyzed overlap.

In order to determine whether an acoustic stimulation can be performed, the artificial intelligence predicts an effect that would produce the potential acoustic stimulation if it were performed at moment a1, a2 of the potential acoustic stimulation.

“Moment of the potential acoustic stimulation” is understood to mean a moment which is not fixed in time. In particular, if an acoustic stimulation is actually carried out, the moment when the acoustic stimulation is carried out may be different from the moment of the potential acoustic stimulation.

This may in particular be due to the absence of a slow wave in the signal S at the moment of the potential acoustic stimulation. It is then possible to wait for the appearance of a slow wave before performing the acoustic stimulation.

The effect may be:
an awakening of the individual P,
a non-awakening of the individual P,
a changing of the sleep phase of the individual P.

The effect may also comprise an evolution in the sleep phase of the individual P.

In particular, the effect may be:
the appearance of slow waves in the EEG of the individual P,
the multiplication of slow waves in the EEG of the individual P,
the increase in the amplitude of the slow waves in the EEG of the individual P,
the generation of spindles,
the increase in the density of spindles.

In addition, the acoustic stimulation may cause an absence of an effect, meaning that the acoustic stimulation will have no impact on the individual.

As described above, the artificial intelligence may be a neural network. The neural network is trained to detect the effect of an acoustic stimulation, during a prior learning step.

A neural network is a system whose design is inspired by the functioning of biological neurons. Neural networks are optimized by learning methods, in particular probabilistic learning.

The neural network receives data as input, with which a weight can be associated. Through learning, the neural network converges to obtain the desired information as output.

Here, the input data are the measurement signals of the individual P, and the information obtained as output from the neural network is the effect induced by a potential acoustic stimulation.

More specifically, the neural network can be supplied the measurement signals S acquired at least before and after an acoustic stimulation of the individual P.

According to one embodiment, the measurement signals S are also acquired during the acoustic stimulation.

In particular, the measurement signals of the individual P comprise at least one or more of: a cardiac activity signal, movements, an EEG signal, and/or a breathing signal from the individual P.

The measurement signals may be processed as described above or the measurement signals may be unprocessed, in other words raw.

The neural network may also be supplied with physiological data entered by the user via the input device of the acquisition means 3.

The neural network may also be supplied with the user's past data. In particular, the neural network may be supplied with the data collected during the previous nights. These data comprise, for example, the effect of simulations carried out on previous nights.

The neural network may also be supplied with the data collected during the night. These data comprise, for example, the effect of simulations carried out previously, or the number of simulations already performed.

The neural network can therefore be trained on the basis of a multitude of measurement signals from the individual P. This makes it possible, on the one hand, to increase the robustness of the neural network, since the effect of the acoustic stimulation can be predicted on the basis of a plurality of measurement signals. Also, this makes it possible to predict the effect of an acoustic stimulation on the basis of a single type of measurement signal (EEG, EOG, cardiac activity signal, movements, breathing, etc.).

The neural network comprises for example several convolutional layers coupled to rectified linear functions and to compression operations. This makes it possible to extract the data useful for learning and for prediction by the neural network, from raw signals or from raw physiological data supplied to the neural network.

The neural network is also able to make this prediction automatically.

In particular, the neural network can be coupled to a predictor responsible for making the prediction. The predictor may be a neural network, a support-vector machine, a decision tree, a set-based model, etc.

The neural network can be trained on the basis of 100,000 samples, divided in equivalent proportions between training samples and test samples, for example a 1:1 proportion.

The duration of training such a neural network is for example about an hour on a graphics processing unit.

Ultimately, the use of a neural network makes it possible to limit human intervention in the prediction process, or even to eliminate any human intervention. The number of errors in predicting an effect is thus greatly reduced, or even zero.

A change between the measurement signal S acquired before the acoustic stimulation and the measurement signal S acquired after the acoustic stimulation can be detected. This change can then be associated with a predefined effect.

This is more precisely illustrated in FIG. 6, in which a measurement signal S of an individual is acquired. The neural network receives for example section s3 of the measurement signal S as input. Section s3 of the measurement signal S corresponds to a section of the measurement signal S acquired during acquisition duration d3 defined above.

An acoustic stimulation A is then performed at time t, after the end of acquisition duration d3.

The measurement signal S of the individual is then analyzed for an analysis duration r3. This therefore corresponds to the analysis of a second section s′3 of the measurement signal S, acquired during the analysis duration r3. From section s′3, the effect generated by the acoustic stimulation A can be determined.

The neural network is trained from these data until it reaches a threshold of convergence.

This threshold of convergence that can be used is the F1-score. For example, a threshold of convergence greater than an F1-score of 0.65 may be chosen, and preferably greater than or equal to 0.75.

Then, the neural network is able to predict the effect of an acoustic stimulation on the individual P by analyzing a measurement signal S acquired in the time interval preceding an acoustic stimulation. From the result of the prediction, the emission means 4 emit or do not emit an acoustic signal audible to the individual P, as described below.

In another embodiment of the invention, possibly combinable with the embodiment detailed above, the prior learning step of the neural network is accomplished by the acquisition of a plurality of measurements signals from a plurality of individuals.

More specifically, a plurality of individuals is equipped with a device 1 as described. The acquisition means 3 of each device 1 are capable of acquiring measurement signals S from each of the individuals of the plurality of individuals.

More specifically, measurement signals S from a plurality of individuals are acquired at least before and after an acoustic stimulation of the brain waves of each of the individuals.

According to one embodiment, the measurement signals S are also acquired during the acoustic stimulation.

In particular, for each individual among the plurality of individuals, the measurement signals comprise at least one or more among: a cardiac activity signal, movements, an EEG signal, and/or a breathing signal.

The measurement signals may be processed as described above or the measurement signals may be unprocessed, in other words raw.

The neural network may also be supplied with physiological data entered by each individual via the input device of the acquisition means 3.

For each measurement signal, a change between the measurement signal S acquired before the acoustic stimulation and the measurement signal S acquired after the acoustic stimulation may be detected. This change can then be associated with a predefined effect.

On the basis of these data, the neural network is trained until it converges.

The measurement signals thus acquired from the plurality of individuals can be stored. For example, the measurement signals from the plurality of individuals are grouped together in the form of a database 8, which itself is stored.

According to one embodiment, the database 8 is stored on a server 9 that is remote from the device 1. The device 1 and the server 9 form a system 10 for predicting an effect of an acoustic stimulation of the brain waves of an individual.

Advantageously, the database 8 also comprises the measurement signals S from the individual P.

The processor 5 of the device 1 is for example in communication with the server 9, via communication means 11.

According to one embodiment, the communication means 11 may comprise a wireless communication module, for example a module implementing a protocol such as Bluetooth and/or WiFi.

The server 9 may also include data transmission means 12. The communication means 11 of the device 1 and the data transmission means 12 of the remote server 9 are able to communicate with each other, directly (point-to-point communication) or by means of a wide area network such as the Internet.

More specifically, the communication means 11 of the device 1 and the data transmission means 12 of the remote server 9 are capable of exchanging data.

Thus, the communication means 11 of the device 1 may in particular be able to transfer the measurement signals S acquired by the acquisition means 3, to the data transmission means 12 of the remote server 9.

Similarly, the neural network may have access to the measurement signals S acquired by acquisition means of other devices on other individuals, and which are stored in the database 8 of the server 9.

Advantageously, the neural network is trained not only with the measurement signals S from the individual P, but also with the measurement signals S from the plurality of individuals, comprised in the database. The robustness of the neural network is thus greatly improved.

In addition, as the neural network is also trained with the measurement signals S from the individual P, the neural network is personalized for the individual P.

According to the embodiment of FIG. 4, the system 10 comprises the server 9 and a plurality of devices 1.

The plurality of devices 1 communicates with the server 9 via their respective communication means 11. In particular, the measurement signals S acquired on each of the individuals provided with a device 1 among the plurality of devices 1 are stored in the database 8. Thus, each neural network is continuously supplied with measurement signals S.

Alternatively, the neural network may be stored on the server 9.

Finally, the emission means 4 may emit an acoustic signal, depending on the result of the prediction of the effect of an acoustic stimulation.

To this end, the emission means 4 are designed to emit an acoustic signal, audible to the individual, in soft real time.

“Audible” is understood to mean an acoustic signal which can be perceived by the individual P when he is asleep.

“Soft real time” is understood to mean an implementation of the stimulation operation such that the time constraints on this operation, especially on the duration or frequency of repetition of this operation, are met on the average over a total predefined implementation duration, for example a few hours. It is understood in particular that the implementation of said operation may at certain moments exceed said time constraints as long as the average operation of the device 1 and the average implementation of the method satisfies them over the total predefined implementation duration. In particular, time limits may be predefined, beyond which the implementation of the stimulation operation must be stopped or paused.

The acoustic means 4 may comprise at least one acoustic transducer 13 and an electronic control unit 14.

The acoustic transducer(s) 13 are capable of emitting an acoustic signal stimulating at least one inner ear of the individual P.

For example, the acoustic transducer 13 is an osteophonic device stimulating the inner ear of the individual P by bone conduction.

The acoustic transducer 13 may also be a speaker stimulating the inner ear of the individual P through an ear canal leading to the inner ear of the individual P.

The acoustic signal is a modulated signal that is at least partially within a frequency range audible to an individual P.

The electronic control unit 14 is in particular capable, in soft real time, of communicating with the processor 5 to control the emission by the acoustic transducer 13 of an acoustic signal, audible to the individual, depending on a result of the prediction.

As described above, the acoustic stimulation may not be performed immediately after obtaining the result of the prediction. The acoustic stimulation may be performed so as to have the greatest impact on the individual P. More specifically, the acoustic stimulation may be performed in order to be synchronized with a predefined temporal pattern of a slow brain wave of the individual P.

Indeed, the described method makes it possible in particular to stimulate the brain waves of the individual, and more precisely the slow brain waves of the individual P.

“Slow brain wave” is understood in particular to mean an electrical brain wave of the individual P having a frequency of less than 5 Hz and greater than 0.3 Hz. “Slow brain wave” can be understood to mean an electrical brain wave of the individual P having a peak-to-peak amplitude of, for example, between 10 and 200 microvolts. In addition to waves of very low frequencies below 1 Hz, slow brain wave can also be understood in particular to mean delta waves of higher frequencies (usually between 1.6 and 4 Hz). Slow brain wave can also be understood to mean any type of wave having the frequency and amplitude characteristics mentioned above. For example, phase 2 sleep waves called “K-Complexes” can be considered as slow brain waves.

In general, the implementation of the method may for example take place during a sleep phase of the individual P (as identified for example in the standards of the AASM, acronym for “American Academy of Sleep Medicine”), for example a deep sleep phase of the individual P (commonly called stage 3 or stage 4) or during other sleep phases, for example during a light sleep of the individual (usually called stage 2).

The method may also be implemented during an awake phase, or a falling asleep or awakening phase of the individual P. The brain waves can then differ from slow brain waves.

To carry out the acoustic stimulation of the brain waves, the electronic control unit 14 is for example able, from the measurement signal S, to first determine a temporal form F of the slow brain wave C such as is illustrated in FIG. 7.

In a first embodiment, the temporal form F is a series of sampled points of amplitude values of the measurement signal S, possibly preprocessed as mentioned above, said series of measurement points optionally being interpolated or resampled.

In a second embodiment, the temporal form F is a series of amplitude values generated by a phase-locked loop (commonly known as PLL).

The phase-locked loop is such that the instantaneous phase of the temporal form F at the output of said loop is related to the instantaneous phase of the measurement signal S.

The phase-locked loop can be implemented by analog means or digital means.

It is therefore understood that the temporal form F is a representation of the brain wave C which can be directly obtained or can be obtained by a phase-locked loop which makes it possible to obtain a cleaner signal. In particular, the instantaneous phase of the temporal form F and the brain wave C are time-synchronized. In the present description, the term “brain wave C” is therefore used where appropriate to mean the values taken by the temporal form F.

From this temporal form F, the electronic control unit 14 is capable of determining at least one time instant I of synchronization between a predefined temporal pattern M1 of the slow brain wave C and a predefined temporal pattern M2 of the acoustic signal A.

Then, the electronic control unit 14 is able to control the acoustic transducer 13 so that the predefined temporal pattern M2 of the acoustic signal A is emitted at the time instant I of synchronization.

The predefined temporal pattern M1 of the slow brain wave C is therefore a pattern of amplitude and/or phase values of the temporal form F which represents the slow brain wave C. In particular, the predefined temporal pattern M1 can be a succession of phase values of the temporal form F and can therefore in particular be independent of the absolute value of the amplitude of the temporal form F.

The predefined temporal pattern M1 can also be a succession of relative values of the amplitude of the temporal form F. Said relative values are for example relative to a predefined or stored maximum amplitude of the temporal form F.

In one embodiment of the invention, the predefined temporal pattern M1 can thus for example correspond to a local temporal maximum of the slow brain wave C, a local temporal minimum of the slow brain wave C, or else a predefined succession of at least one local temporal maximum and at least one local temporal minimum of the slow brain wave C.

The predefined temporal pattern M1 may also correspond to a portion of such a maximum or minimum or of such a succession, for example a rising edge, a falling edge, or a plateau.

In the same manner, the predefined temporal pattern M2 of the acoustic signal may be a pattern of amplitude and/or phase values of the acoustic signal A.

In a first embodiment, the acoustic signal is for example an intermittent signal as illustrated in FIG. 7. This intermittent signal is for example emitted for a duration that is less than a period of a slow brain wave. The duration of the intermittent signal is for example less than a few seconds, preferably less than one second.

In an example given purely as a non-limiting example, the acoustic signal A is for example a pulse of 1/f type pink noise having a duration of 50 to 100 milliseconds with a rise time and fall time of a few milliseconds. Again in a non-limiting manner and to illustrate the concept, in this example the predefined temporal pattern M1 of the slow brain wave C may for example correspond to a rising edge of a local maximum of the slow brain wave C. The predefined temporal pattern M2 of the acoustic signal A can then for example be a rising edge of the pink noise pulse. In this example, the time instant I of synchronization between the predefined temporal pattern M1 of the slow brain wave C and the predefined temporal pattern M2 of the acoustic signal A may for example be defined such that the rising edge of the pink noise pulse A and the rising edge of the local maximum of the slow brain wave C are synchronized, in other words concomitant.

In another embodiment, the acoustic signal A may be a continuous signal. The duration of the acoustic signal A can then in particular be greater than a period of the slow brain wave C. “Continuous signal” is understood in particular to mean a signal of great duration than a period of the slow brain wave C.

In this embodiment, the acoustic signal A may be time-modulated in amplitude, frequency, or phase, and the predefined temporal pattern M2 of the acoustic signal A can then be such a time-modulation.

Alternatively, the continuous acoustic signal A may be not time-modulated, for example in a manner which will now be described.

The device 1 may comprise at least two acoustic transducers 13, in particular a first acoustic transducer 13a and a second acoustic transducer 13b as illustrated in FIG. 3. The first acoustic transducer 13a is capable of emitting an acoustic signal stimulating a right inner ear of the individual P. The second acoustic transducer 13b is capable of emitting an acoustic signal stimulating a left inner ear of the individual P.

It is then possible in particular to control the first and second acoustic transducer 13a, 13b so that the acoustic signals respectively emitted by each transducer are binaural acoustic signals. To this end, these acoustic signals may for example be continuous signals of different frequencies.

Such acoustic signals are known for generating intermittent pulses in the brain of the individual P, in particular called binaural beats.

Still without limitation and to illustrate the concept, in this example the predefined temporal pattern M1 of the slow brain wave C may for example, again, correspond to a rising edge of a local maximum of the slow brain wave C. The predefined temporal patterns M2 of the acoustic signals emitted by the acoustic transducers 13a, 13b may moreover be ranges of the acoustic signals temporally corresponding to said intermittent pulses generated in the brain of the individual P. In this example, the time instant I of synchronization between the predefined temporal pattern M1 of the slow brain wave C and the predefined temporal patterns M2 of these acoustic signals may for example be defined such that an intermittent pulse generated in the brain of the individual P is temporally synchronized with the rising edge of the local maximum of the slow brain wave C.

FIG. 7 illustrates an example of predefined temporal patterns M1 and M2.

One or more among a sound level, a duration, a spectrum, and a temporal pattern M2 of the acoustic signal A may be predefined and stored in the memory 6 of the device 1.

As stated above, the electronic control unit 14 controls the emission of an acoustic signal A by at least one acoustic transducer 13, according to the result of the prediction of the effect of the acoustic stimulation. More specifically, the electronic control unit 14 controls the emission of an acoustic signal by the acoustic transducer 13 according to the effect predicted by the neural network on the basis of the measurement signal S of the individual P, acquired and analyzed by said neural network.

As a non-limiting example, when the neural network predicts that the acoustic stimulation will generate the “awakening of individual P” effect, the electronic control unit 14 does not order the emission of the audible acoustic signal by the individual P.

Alternatively, at least one parameter of the acoustic signal A may be modified by the electronic control unit 14. The modified acoustic signal A can be emitted during an upcoming acoustic stimulation.

In particular, a parameter of the acoustic signal may comprise a sound volume, a frequency, a duration, and/or an acoustic pattern.

When the neural network predicts that the acoustic stimulation will not generate any effect, the electronic control unit 14 is for example capable of modifying at least one parameter of the acoustic signal audible to the individual P, before emission of this acoustic signal in order to generate an effect.

In particular, a parameter of the acoustic signal may comprise a sound volume, a frequency, a duration, and/or an acoustic pattern.

According to an alternative embodiment, the electronic control unit 14 may not order the emission of the acoustic signal audible to the individual P. At least one parameter of the acoustic signal is modified by the electronic control unit 14. The modified acoustic signal can be emitted during the next acoustic stimulation.

When the neural network predicts that the acoustic stimulation will generate one or more of the following effects: “appearance of slow waves in the EEG of the individual P”, “multiplication of slow waves in the EEG of the individual”, “increase in the amplitude of the slow waves in the EEG of the individual P”, or “changing of the sleep phase of the individual P”, the electronic control unit 14 orders the emission of an acoustic signal audible to the individual P.

According to one embodiment, the neural network predicts that the acoustic stimulation will generate one or more of the following effects: “appearance of slow waves in the EEG of the individual P”, “multiplication of slow waves in the EEG of the individual P”, “increase in the amplitude of the slow waves in the EEG of the individual P”, or “changing of the sleep phase of the individual P”. An acoustic stimulation can therefore be performed.

However, a parameter of the acoustic signal A may be modified in order to amplify one or more of these effects. In this case, the electronic control unit 14 is for example capable of modifying at least one parameter of the acoustic signal audible to the individual P, before emission of this acoustic signal, in order to amplify the effect.

Reference:

device 1
support member 2
acquisition means 3
emission means 4
processor 5
memory 6
battery 7
database 8
server 9
system 10
communication means 11
data transmission means 12
acoustic transducer 13
electronic control unit 14

1. A method, implemented by computer means, for predicting an effect of an acoustic stimulation of the brain waves of an individual, the method comprising:
acquisition of at least one measurement signal representative of a physiological signal of the individual, by a device for acoustic stimulation of brain waves that is suitable for being worn by the individual,
analysis of said measurement signal by an artificial intelligence trained to predict the effect of an acoustic stimulation, and
determination of whether an acoustic stimulation is to be performed by the device.

2. The method according to claim 1, wherein the artificial intelligence is a neural network, the method including a prior learning step comprising:
a plurality of successive acoustic stimulations of the brain waves of said individual,
an acquisition of measurement signals representative of a physiological signal of said individual at least before and after each of the acoustic stimulations of said plurality of acoustic stimulations,
for each of the acoustic stimulations, determination of a change in the measurement signals acquired after an acoustic stimulation in comparison to the measurement signal acquired before this acoustic stimulation, and association of an effect with said change,
training of said neural network until a threshold of convergence is reached, and
storage of said neural network.

3. The method according to claim 2, wherein the learning step further comprises the indication of physiological data of said individual, the neural network being further trained to predict the effect of an acoustic stimulation on the basis of said physiological data of said individual.

4. The method according to claim 1, wherein the artificial intelligence is a neural network, the method including a prior learning step comprising:
at least one acoustic stimulation of the brain waves of a plurality of individuals,
an acquisition of measurement signals representative of a physiological signal of said plurality of individuals at least before and after said acoustic stimulation,
determination of a change in the measurement signals acquired after said acoustic stimulation in comparison to the measurement signals acquired before said acoustic stimulation, and association of an effect with said change,
training of said neural network until a threshold of convergence is reached, and
storage of said neural network.

5. The method according to claim 4, wherein the learning step further comprises the indication of physiological data of said plurality of individuals, the neural network being further trained to predict the effect of an acoustic stimulation on the basis of said physiological data of said plurality of individuals.

6. The method according to claim 2, wherein measurement signals representative of a physiological signal of said individual are acquired continuously, the neural network also being trained continuously and in real time to predict the effect of an acoustic stimulation on the basis of said measurement signals of said individual.

7. The method according to claim 1, wherein an acoustic stimulation comprises the emission of an acoustic signal, the method further comprising a changing of at least one parameter of the acoustic signal to be emitted, on the basis of a result of the prediction by the artificial intelligence of the effect of an acoustic stimulation.

8. A device for predicting an effect of an acoustic stimulation of the brain waves of an individual, comprising:
an acquisition module configured to acquire at least one measurement signal representative of a physiological signal of the individual,
a processor communicating with the acquisition module and configured to analyze said at least one measurement signal representative of a physiological signal of the individual, by an artificial intelligence trained to predict the effect of an acoustic stimulation.

9. The device according to claim 8, further comprising:
an emission module configured to emit an acoustic signal audible to the individual, and communicating with said processor, said acoustic signal being emitted or not emitted depending on a result of the prediction by the artificial intelligence of the effect of said acoustic stimulation.

10. The device according to claim 8, wherein the artificial intelligence comprises a neural network trained to predict the effect of an acoustic stimulation and wherein the device for acoustic stimulation further comprises a memory storing said neural network.

11. A system for predicting an effect of an acoustic stimulation of the brain waves of an individual, comprising:
a device for predicting an effect of an acoustic stimulation of the brain waves of an individual, comprising:
an acquisition module configured to acquire at least one measurement signal representative of a physiological signal of the individual,
a processor communicating with the acquisition module and configured to analyze said at least one measurement signal representative of a physiological signal of the individual, by an artificial intelligence trained to predict the effect of an acoustic stimulation,
a server that is remote from said device.

12. The system according to claim 11, wherein the server is configured to store a database comprising a plurality of measurement signals representative of a physiological signal of at least one individual, said plurality of measurement signals having been acquired by said device.

13. The system according to claim 11, comprising a plurality of devices, the plurality of devices being in communication with the server.

14. The method according to claim 2, wherein the artificial intelligence is a neural network, the method including a prior learning step comprising:
at least one acoustic stimulation of the brain waves of a plurality of individuals,
an acquisition of measurement signals representative of a physiological signal of said plurality of individuals at least before and after said acoustic stimulation,
determination of a change in the measurement signals acquired after said acoustic stimulation in comparison to the measurement signals acquired before said acoustic stimulation, and association of an effect with said change,
training of said neural network until a threshold of convergence is reached, and
storage of said neural network.

15. The method according to claim 3, wherein the artificial intelligence is a neural network, the method including a prior learning step comprising:
at least one acoustic stimulation of the brain waves of a plurality of individuals,
an acquisition of measurement signals representative of a physiological signal of said plurality of individuals at least before and after said acoustic stimulation,
determination of a change in the measurement signals acquired after said acoustic stimulation in comparison to the measurement signals acquired before said acoustic stimulation, and association of an effect with said change,
training of said neural network until a threshold of convergence is reached, and
storage of said neural network.

16. The method according to claim 3, wherein measurement signals representative of a physiological signal of said individual are acquired continuously, the neural network also being trained continuously and in real time to predict the effect of an acoustic stimulation on the basis of said measurement signals of said individual.

17. The method according to claim 4, wherein measurement signals representative of a physiological signal of said individual are acquired continuously, the neural network also being trained continuously and in real time to predict the effect of an acoustic stimulation on the basis of said measurement signals of said individual.

18. The method according to claim 5, wherein measurement signals representative of a physiological signal of said individual are acquired continuously, the neural network also being trained continuously and in real time to predict the effect of an acoustic stimulation on the basis of said measurement signals of said individual.

19. The method according to claim 2, wherein an acoustic stimulation comprises the emission of an acoustic signal, the method further comprising a changing of at least one parameter of the acoustic signal to be emitted, on the basis of a result of the prediction by the artificial intelligence of the effect of an acoustic stimulation.

20. The method according to claim 3, wherein an acoustic stimulation comprises the emission of an acoustic signal, the method further comprising a changing of at least one parameter of the acoustic signal to be emitted, on the basis of a result of the prediction by the artificial intelligence of the effect of an acoustic stimulation.
